(19) United States
(12) Patent Application Publication
(10) Pub. No.: US 2008/0086092 A1
(43) Pub. Date: Apr. 10, 2008

(54) Syringe shield

(76) Inventor: Thomas W. Loe, Dallas, TX (US)

Correspondence Address: John M. Harrison, 2139 E. Bert Kouns, Shreveport, LA, 71105, US

(21) Appl. No.: 11/906,862

(22) Filed: Oct. 4, 2007

Related U.S. Application Data
(60) Provisional application No. 60/850,303, filed on Oct. 10, 2006.

(51) Int. Cl. A61M 5/32; G21F 5/00

(52) U.S. Cl. 604/198; 250/506.1

(57) ABSTRACT

A syringe shield for protecting a user from radiation emanating from a radioactive liquid disposed within an enclosed syringe. In a preferred embodiment the syringe shield includes a tungsten component housing or barrel fitted with a yieldable grip and a lead glass window for receiving the vial of a syringe containing the radioactive material. A barrel lock fits over the syringe vial and engages the syringe entry end of the barrel, such that resilient fingers provided on the barrel lock engage a barrel taper in the barrel and bend inwardly from an extended configuration to grip the syringe vial and maintain the syringe in position inside the barrel. The barrel lock can be friction-fitted or threaded in the receiving end of the barrel or it may be provided with a bayonet coupling pin which engages a companion bayonet slot in the barrel to close the barrel lock on the barrel such that the fingers tightly, yet removably, engage the syringe vial in a compression fit. Alternatively, the syringe can be secured inside the barrel using a lock nut which is tightened on a tapered, slotted barrel nipple extending from the entry end of the barrel.

### CROSS-REFERENCE TO RELATED APPLICATIONS

This application claims the benefit of and incorporates by reference copending Provisional Application Ser. No. 60/850,303, Filed Oct. 10, 2006.

### FIELD OF THE INVENTION

This invention relates to devices for protecting users and handlers from radiation emitted by liquid radioactive materials in syringes, typically during medical procedures. More particularly, the invention relates to a syringe shield for protecting the users and handlers from radiation emanating from a liquid radioactive material disposed within an enclosed syringe vial. The syringe shield of this invention is characterized in a one embodiment by a cylindrical tungsten, tungsten-filled nylon and/or tungsten and barium-filled nylon barrel for receiving the syringe vial, and a barrel lock is designed to fit over the syringe vial and engage the entry end of the barrel to retain the syringe vial in the barrel. This action forces multiple, resilient fingers provided on one end of the barrel lock against the syringe vial in the barrel and secures the syringe inside the barrel in a compression fit. The syringe-receiving end of the barrel is typically fitted with one or more grooves or threads for receiving a corresponding thread or threads provided on the barrel lock and removably securing the barrel lock to the barrel. Alternatively, the syringe is secured inside the barrel by a lock nut that threads onto a tapered, slotted nipple extending from the syringe-entry end of the barrel. In still another embodiment a bayonet slot is provided at the receiving end of the barrel for receiving a bayonet pin provided on the barrel lock to removably secure the barrel lock on the barrel. In the barrel lock embodiment described above the housing barrel is provided with a tapered interior or throat at the syringe and barrel lock-receiving end, for receiving the correspondingly, shaped, spaced-apart barrel lock fingers as the fingers move into the barrel. There lock fingers engage the throat taper and bend inwardly against the syringe vial to secure the syringe vial inside the barrel. A leaded acrylic or glass window is typically provided in the barrel for viewing the syringe vial contents.

### BRIEF DESCRIPTION OF THE DRAWINGS

The invention will be better understood by reference to the accompanying drawings, wherein:

FIG. 1 is a perspective view of a barrel lock embodiment of the syringe shield of this invention with the syringe vial of a syringe mounted therein, which syringe is illustrated in phantom lines;

FIG. 2 is an exploded view of the syringe shield illustrated in FIG. 1 and the syringe in phantom lines, more particularly illustrating a preferred threaded embodiment of the barrel lock element;

FIG. 3 is a sectional view taken along line 3-3 of the syringe shield illustrated in FIG. 1;

FIG. 4 is a sectional view taken along line 4-4 of the syringe shield illustrated in FIG. 3, more particularly illustrating the threaded barrel lock illustrated in FIGS. 2 and 3 in functional position for threadably engaging the receiving end of the barrel;

FIG. 5 is a side view, partially in section, of the receiving end of the barrel, more particularly illustrating an embodiment of the barrel lock fitted with external threads for engaging internal threads provided in the receiving end of the barrel;

FIG. 6 is a longitudinal sectional view of the barrel, illustrating another embodiment wherein the barrel lock is fitted with a single or monothread and the receiving end of the barrel provided with a single monogroove for receiving the monothread on the barrel lock;

FIG. 7 is a longitudinal sectional view of the barrel fitted with a bayonet slot for receiving a corresponding bayonet pin provided on the barrel lock element;

FIG. 8 is a perspective view of a lock nut embodiment of the syringe shield with the syringe vial of a syringe mounted therein;

FIG. 9 is an exploded view of the syringe shield barrel with the lock nut removed; and

FIG. 10 is a sectional view taken along line 10-10 of the syringe shield and the enclosed syringe.

### DESCRIPTION OF THE PREFERRED EMBODIMENTS

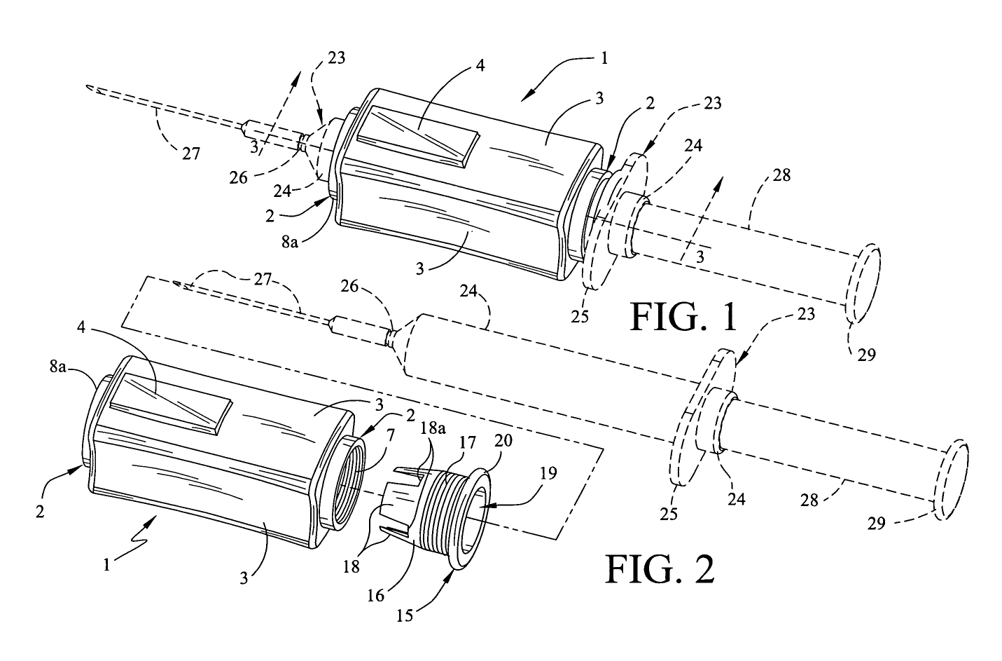

Referring initially to FIGS. 1 and 2 of the drawings, in a first embodiment the syringe shield of this invention is generally illustrated by reference numeral 1. The syringe shield 1 is characterized by a cylindrical barrel 2, typically having a deformable, shaped barrel grip 3 of selected thickness and material, typically durometer 80A rubber, on the outside thereof. The barrel 2 is typically further provided with a lead glass window 4 for viewing the contents of the syringe vial 24 of a syringe 23, illustrated in phantom, which is inserted in the barrel 2. The syringe vial 24 is positioned longitudinally inside the barrel 2 of the syringe shield 1 to shield the radiation emitted from a radioactive liquid (not illustrated) contained in the syringe vial 24. The syringe 23 is typically conventional in design, wherein the syringe vial 24 is of conventional size and length, having a vial flange 25 and a plunger 28 extending through the vial flange 25 and into one end of the syringe vial 24. The syringe vial 24 is further fitted with a needle mount 26 on the end opposite the vial flange 25, for receiving a needle 27 in conventional fashion. A plunger flange 29 is typically provided on the extending end of the plunger 28 for accommodating the thumb, while two fingers of the hand are normally placed around the vial flange 25 to facilitate depression of the plunger 28 in the syringe vial 24 and distribution of the liquid contents of the syringe vial 24 through the hollow needle 27 into the body of a patient, in conventional fashion. One or more barrel threads 7 are typically provided internally in the receiving end of the barrel 2, as illustrated in FIG. 2.

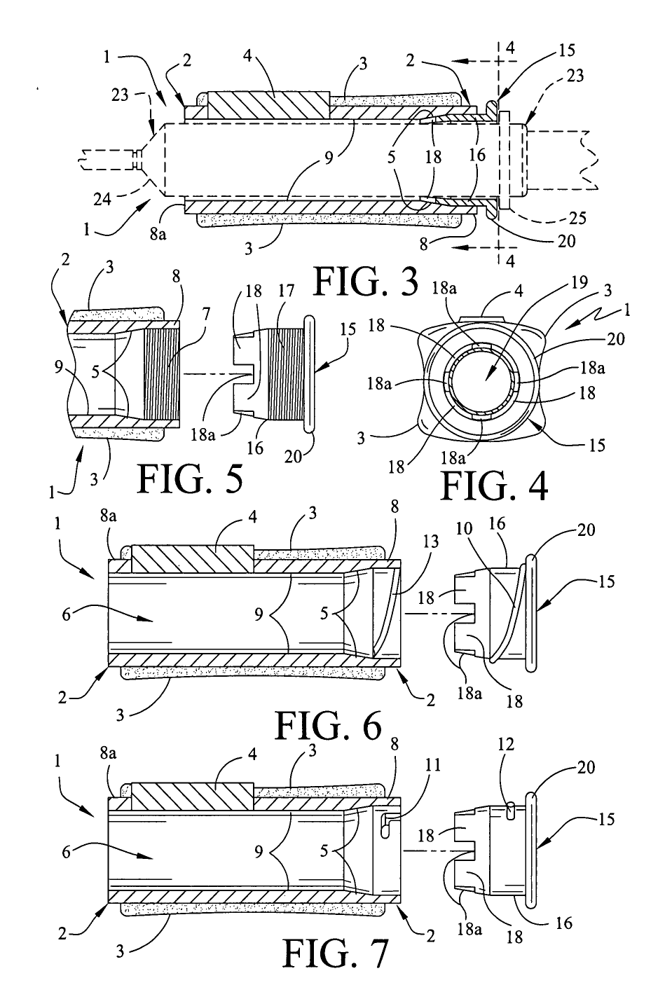

As further illustrated in FIGS. 2, 4 and 5 of the drawings, in a typical embodiment a barrel lock 15 is typically characterized by a cylindrical barrel lock sleeve 16, typically fitted with external sleeve threads 17 and terminated at one end by multiple sleeve fingers 18, which may be spaced-apart by finger slots 18a. A central sleeve opening 19 is provided in the barrel lock sleeve 16 for accommodating the syringe vial 24 of the syringe 23 and the opposite end of the barrel lock 15 from the sleeve fingers 18 typically defines a sleeve flange 20, as further illustrated in FIG. 2 of the drawings. The barrel lock 15 is designed to fit on the threaded end 8 of the barrel 2 such that the sleeve threads 17 of the barrel lock 15 engage the corresponding internal barrel threads 7 of the barrel 2 to removably retain the barrel lock 15 on the barrel 2 at the threaded end 8. Alternatively, the barrel lock sleeve 16 can be designed sans threads, for a tight, removable friction-fit in the barrel 2 (FIG. 3).

Referring again to FIGS. 3 and 5 of the drawings, the typically threaded end 8 of the barrel 2 is also provided with an internal throat or barrel taper 5 that extends from a maximum diameter at the barrel threads 7 of the threaded end 8 into the barrel bore 6 and terminates in a minimum diameter at the inside barrel wall 9. The barrel taper 5 is designed to receive and bend the spaced-apart, flexible, resilient and typically tapered sleeve fingers 18, responsive to threadable engagement of the barrel lock 15 with the threaded end 8 of the barrel 2. Accordingly, this engagement causes the sleeve fingers 18 to bend inwardly in concert inside the barrel bore 6 and seat against the syringe vial 24 of the syringe 23, in a compression action, as illustrated in FIG. 1. Consequently, when the barrel lock 15 is snugly friction-fitted or threaded on the threaded end 8 of the barrel 2, the respective sleeve fingers 18 are caused to securely engage and grip the syringe vial 24 of the syringe 23 and maintain the syringe vial 24 of the syringe 23 inside the barrel 2 of the syringe shield 1.

Referring now to FIG. 6 of the drawings, in another embodiment of the invention the threaded end 8 of the barrel 2 is fitted with a monogroove 13 and the barrel lock sleeve 16 of the barrel lock 15 is provided with a correspondingly shaped monothread 10, for engaging the monogroove 13 and removably securing the barrel lock 15 on the threaded end 8 of the barrel 2 with a twist of the barrel lock 15.

In yet another embodiment of the invention illustrated in FIG. 7 of the drawings, at least one L-shaped bayonet slot 11 is provided in the unthreaded or threaded end 8 of the barrel 2 and is designed to accommodate a corresponding bayonet pin 12, extending from the barrel lock sleeve 16 of the barrel lock 15. Accordingly, when the barrel lock sleeve 16 and sleeve fingers 18 illustrated in FIG. 7 are inserted in the unthreaded or threaded end 8 of the barrel 2, the bayonet pin 12 is aligned with and seated in the corresponding bayonet slot 11 and the barrel lock 15 is then rotated to tightly engage the bayonet pin 12 in the bayonet slot 11 and removably secure the barrel lock sleeve 16 inside the unthreaded or threaded end 8 of the barrel 2. This action also tightens the sleeve fingers 18 on the syringe vial 24 in a compression fit, as described above.

Referring again to FIGS. 1-5 of the drawings, in use, the illustrated barrel lock embodiment of the syringe shield 1 is first deployed as illustrated in FIG. 2, with the barrel lock 15 either disengaged from or loosened in the threaded end 8 of the barrel 2, to facilitate insertion of the needle 27 and the syringe vial 24 of the syringe 23 through the sleeve opening 19 of the barrel lock 15, past the sleeve fingers 18 and into the aligned barrel bore 6 of the barrel 2, to the position illustrated in FIG. 1. The barrel lock 15 is then tightened on the threaded end 8 of the barrel 2 by engaging the sleeve threads 17 with the corresponding internal barrel threads 7. Tightening of the barrel lock 15 on the threaded end 8 of the barrel 2 causes the respective sleeve fingers 18 to engage the barrel taper 5 (FIG. 5) and bend inwardly toward each other in concert against the syringe vial 24 of the syringe 23, to tightly, yet removably, secure the syringe vial 24 of the syringe 23 inside the barrel 2 of the syringe shield 1.

Referring again to FIG. 3, it will be appreciated that the threaded end 8 of the barrel 2 need not be threaded but can be smooth, but still fitted with the barrel taper 5 to accommodate the respective sleeve fingers 18 in a compression action when the barrel lock 15 is tightly inserted in the barrel 2. This action also serves to bend the sleeve fingers 18 inwardly against the barrel taper 5 and tightly engage the syringe valve 24 of the syringe 23.

Similarly, referring to FIG. 6 of the drawings, under circumstances where the threaded end 8 of the barrel 2 is fitted with a single monogroove 13 and the barrel lock sleeve 16 of a barrel lock 15 is provided with a corresponding monothread 10, insertion of the barrel lock 15 in the barrel 2 at the threaded end 8 and engagement of the monothread 10 with the monogroove 13 and twisting of the barrel lock 15 also causes inward bending of the respective sleeve fingers 18 against the syringe vial 24 to secure the syringe 23 inside the barrel 2 of the syringe shield 1.

A like connection is effected in the syringe shield 1 embodiment illustrated in FIG. 7, wherein the threaded end 8 need not be threaded but may be provided with a bayonet slot 11 for engagement with a corresponding bayonet pin 12, when the sleeve fingers 18 are inserted in the threaded end 8 and the bayonet pin 12 is aligned with the bayonet slot 11. Twisting of the barrel lock 15 thus fully engages the bayonet pin 12 with the corresponding L-shaped bayonet slot 11 and secures the spaced-apart sleeve fingers 18 against the corresponding enclosed syringe vial 24 of the syringe 23.

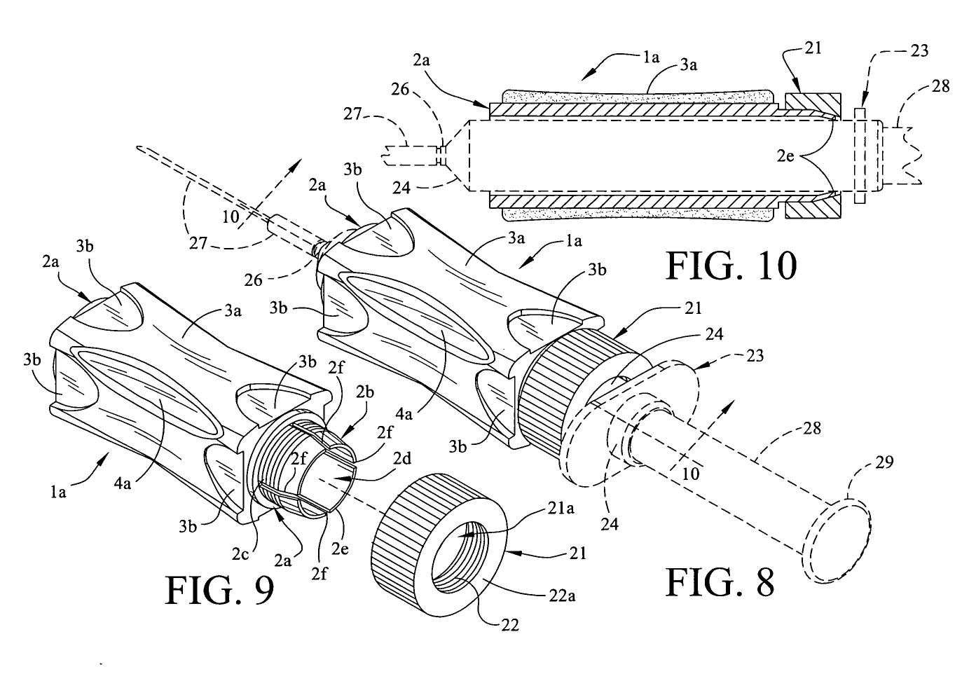

Referring now to FIGS. 8-10 of the drawings, in still another embodiment of the invention a syringe shield 1a is illustrated and is characterized by a cylindrical barrel 2a, fitted with a typically durometer 80 rubber barrel grip 3, having a leaded acrylic or glass window 4a provided therein. In one embodiment multiple grip recesses 3b are provided in the ends of the barrel grip 3a to facilitate better gripping of the syringe shield 1a when the syringe vial 24 (illustrated in phantom) of a syringe 23 is inserted inside the longitudinal bore of the barrel 2a of the syringe shield 1a in the manner illustrated in FIGS. 8 and 10 and in the embodiments illustrated in FIGS. 1-7 of the invention. In the FIGS. 8-10 embodiment, a tapered barrel nipple 2b projects from the syringe-receiving end of the barrel 2a and is characterized by a nipple opening 2d, that communicates with the bore extending through the barrel 2a. The barrel nipple 2b tapers to a nipple rim 2e and includes spaced-apart barrel slots 2f, extending through external nipple threads 2c, as further illustrated in FIG. 9 of the drawings. Like the barrel 2a, the barrel nipple 2b is typically constructed of a resilient material such as plastic and tapers to a thin margin separated by the barrel slots 2f at the nipple rim 2e, as illustrated in FIGS. 9 and 10.

A generally cylindrical lock nut 21 has a lock nut opening 21a that communicates with the nipple opening 2d of the barrel nipple 2b and is also fitted with tapered internal lock threads 22, that terminate inwardly of a lock nut base 22a. Accordingly, when it is desired to enclose the syringe vial 24 of a conventional syringe 23 inside the barrel 2a of the syringe shield 1a, the lock nut 21 is removed from or loosened on the barrel nipple 2b as illustrated in FIG. 9 and the syringe vial 24 is inserted in the lock nut opening 21a, through the nipple opening 2d and extended through the bore of the barrel 2a, to the enclosed position illustrated in FIGS. 8 and 10. The lock nut 21 is then threadably tightened on the barrel nipple 2b by engaging the internal lock nut threads 22 with the external nipple threads 2c to the position illustrated in FIGS. 8 and 10. Tightening of the lock nut 21 on the barrel nipple 2b deforms the slotted nipple rim 2e inwardly sufficiently to contact the syringe vial 24 and securely, yet removably, enclose the syringe vial 24 of the syringe 23 inside the barrel 2a.

Referring again to the drawings, it will be appreciated by those skilled in the art that the barrels 2 and 2a of the respective syringe shields 1 and 1a can be designed and sized to completely enclose a syringe vial 24 of any size in any syringe 23, in order to shield the user from radiation emitted from the radioactive contents of the syringe vial 24. Furthermore, the contents of the syringe vial 24 can be constantly visually monitored through the respective windows 4 and 4a, which are typically constructed of leaded acrylic or glass, to further protect the user from the effects of the radiation therein. This visual monitoring can be important during dispensing of the liquid radioactive material from the syringe vial 24 through the hollow needle 27, as the plunger 28 is depressed in conventional fashion.

It will also be appreciated from a consideration of the drawings that the barrel lock 15 of the syringe shield 1 (FIGS. 1 and 2) and the barrel nipple 2b (FIGS. 9 and 10) should be constructed of a material which is sufficiently resilient to allow one or more sleeve fingers 18 and the slotted nipple rim 2e, respectfully, to depress inwardly as detailed above, and properly secure the syringe vial 24 of the syringe 23 inside the syringe shields 1 and 1a. In the case of the syringe shield 1 this inward depression of the respective sleeve fingers 18 in concert is typically facilitated by the finger slots 18a, which may be of a desired width and number and are located between the sleeve fingers 18, to allow free depression of the sleeve fingers 18 against the syringe vial 24 as the barrel lock 15 tightly engages the barrel 2. Furthermore, removal of the syringe vial 24 and the syringe 23 from the syringe shield 1 is easily accomplished under circumstances where the sleeve fingers 18 are resilient and therefore have a “memory” and are able to return to the original, extended and non-depressed configuration, upon loosening of the barrel lock 15 from the threaded end 8 of the barrel 2. The syringe 23 is therefore quickly and easily removed from the barrel 2 of the syringe shield 1 by simply unthreading or uncoupling the barrel lock 15 from the threaded end 8 of the barrel 2. In like manner, the slotted nipple rim 2e and barrel nipple 2b of the syringe shield are resilient and sized to facilitate inward flexure and tightening on the syringe vial 24 responsive to tightening of the lock nut 21 on the barrel nipple 2b.

While the preferred embodiments of the invention have been described above, it will be recognized and understood that various modifications may be made in the invention and the appended claims are intended to cover all such modifications which may fall within the spirit and scope of the invention.

Having described my invention with the particularity set forth above, what is claimed is:

1. A shield for shielding a user from radioactive material in a syringe, comprising a barrel constructed of a radiation-resistant material for receiving the syringe; a barrel taper provided in one end of said barrel; a barrel lock for seating on said one end of said barrel, said barrel lock having a barrel lock opening for receiving the syringe; and at least one resilient finger provided on said barrel lock for extending into said one end of said barrel and contacting said barrel taper and engaging the syringe responsive to said seating of said barrel lock on said one end of said barrel.

2. The shield of claim 1 comprising at least one barrel groove provided in said one end of said barrel and at least one barrel lock thread provided on said barrel lock for engaging said at least one barrel groove and removably securing said barrel lock on said one end of said barrel and seating said at least one resilient finger against the syringe.

3. The shield of claim 2 wherein said at least one barrel groove comprises a plurality of barrel threads provided in said one end of barrel and said at least one barrel lock thread comprises a plurality of barrel lock threads provided on said barrel lock for engaging said barrel threads and facilitating said seating of said barrel lock on said one end of said barrel.

4. The shield of claim 1 comprising at least one bayonet slot provided in said one end of said barrel and at least one bayonet pin provided on said barrel lock for selectively engaging said bayonet slot and removably securing said barrel lock on said barrel and seating said at least one resilient finger against the syringe.

5. The shield of claim 1 wherein said at least one resilient finger comprises a plurality of resilient fingers provided on said barrel lock for engaging the syringe when said barrel lock is fitted on said one end of said barrel.

6. The shield of claim 5 comprising at least one barrel groove provided in said one end of said barrel and at least one barrel lock thread provided on said barrel lock for engaging said at least one barrel groove and removably securing said barrel lock on said one end of said barrel and seating said resilient fingers against the syringe.

7. The shield of claim 6 wherein said at least one barrel groove comprises a plurality of barrel threads provided in said one end of barrel and said at least one barrel lock thread comprises a plurality of barrel lock threads provided on said barrel lock for engaging said barrel threads and facilitating said seating of said barrel lock on said one end of said barrel.

8. The shield of claim 5 comprising at least one bayonet slot provided in said one end of said barrel and at least one bayonet pin provided on said barrel lock for selectively engaging said bayonet slot and removably securing said barrel lock on said barrel and seating said resilient fingers against the syringe.

9. The shield of claim 1 comprising a radiation-resistant window provided in said barrel for viewing the contents of the syringe while the syringe is inserted in said barrel.

10. The shield of claim 9 comprising at least one barrel groove provided in said one end of said barrel and at least one barrel lock thread provided on said barrel lock for engaging said at least one barrel groove and removably securing said barrel lock on said one end of said barrel and seating said at least one resilient finger against the syringe.

11. The shield of claim 10 wherein:
(a) said at least one resilient finger comprises a plurality of resilient fingers spaced-apart on said barrel lock for engaging the syringe when said barrel lock is fitted on said barrel; and
(b) said at least one barrel groove comprises a plurality of barrel threads provided in said one end of barrel and said at least one barrel lock thread comprises a plurality of barrel lock threads provided on said barrel lock for engaging said barrel threads and facilitating said seating of said barrel lock on said one end of said barrel.

12. The shield of claim 9 wherein said at least one resilient finger comprises a plurality of resilient fingers spaced-apart on said barrel lock for engaging the syringe when said barrel lock is fitted on said barrel and comprising a bayonet slot provided in said one end of said barrel and a bayonet pin provided on said barrel lock for selectively engaging said bayonet slot and removably securing said barrel lock on said barrel and seating said resilient fingers against the syringe.

13. The shield of claim 11 comprising a deformable coating provided on said barrel.

14. A shield for protecting a user from radiation emanating from radioactive material in the syringe vial of a syringe, said shield comprising a barrel constructed of a radiation-resistant material for removably receiving the syringe vial; a barrel nipple having nipple threads provided on one end of said barrel; at least one barrel slot provided in said barrel nipple; and a barrel lock nut having barrel lock nut threads adapted for fitting over the syringe, with said barrel lock nut threads engaging said nipple threads for gripping the syringe vial by said barrel nipple responsive to torque applied to said barrel lock nut.

15. The shield of claim 14 comprising a deformable coating provided on said barrel.

16. The shield of claim 14 comprising a radiation-resistant window provided in said barrel for viewing the contents of the syringe vial while the syringe is inserted in said barrel.

17. The shield of claim 14 wherein said at least one barrel slot comprises a plurality barrel slots provided on said barrel nipple in spaced-apart relationship with respect to each other and comprising a radiation-resistant window provided in said barrel for viewing the contents of the syringe vial while the syringe vial is inserted in said barrel and a deformable coating provided on said barrel to enhance gripping of said barrel by a user.

18. A shield for receiving a syringe having a syringe vial containing a radioactive material, comprising a radiation-resistant barrel for receiving and substantially enveloping the syringe vial and shielding a user from radiation emitted from the radioactive material; a barrel nipple having nipple threads and a nipple taper provided in one end of said barrel; at least one barrel slot provided in said barrel nipple; a barrel lock nut for receiving the syringe vial; and barrel lock nut threads provided on said barrel lock nut for engaging said nipple threads on said barrel nipple and deforming said barrel nipple onto the syringe vial responsive to threadably tightening said barrel lock nut on said barrel nipple.

19. The shield of claim 18 wherein said at least one barrel slot comprises a plurality of barrel slots provided in said barrel nipple in spaced-apart relationship with respect to each other and comprising a radiation-resistant window provided in said barrel for viewing the contents of the syringe vial while the syringe vial is inserted in said barrel and a deformable coating provided on said barrel to enhance gripping of said barrel by a user and comprising a radiation-resistant window provided in said barrel for viewing the contents of the syringe vial while the syringe vial is inserted in said barrel.

20. The shield of claim 19 comprising a deformable coating provided on said barrel to enhance gripping of said barrel by a user.
